(19) United States
(12) Patent Application Publication
(10) Pub. No.: US 2024/0392360 A1
(43) Pub. Date: Nov. 28, 2024

(54) NUCLEIC ACID DETECTION

(71) Applicant: miDiagnostics NV, Heverlee (BE)

(72) Inventors: Geert MEERSSEMAN, Heverlee (BE); Serena ROLLO, Heverlee (BE); Nicolas VERGAUWE, Heverlee (BE)

(21) Appl. No.: 18/694,536

(22) PCT Filed: Sep. 22, 2022
(86) PCT No.: PCT/EP2022/076362

§ 371 Date: Mar. 22, 2024

(30) Foreign Application Priority Data
Sep. 23, 2021 (EP) 21198450.5

(51) Int. Cl. C12Q 1/6851; C12Q 1/6837

(52) U.S. Cl. CPC C12Q 1/6851; C12Q 1/6837

(57) ABSTRACT

A method for detecting at least one target nucleic acid sequence in a sample, comprising adding an RNA polymerase promoter sequence to the target nucleic acid and detecting protons released by transcription activity, e.g. by an RNA polymerase.

### FIELD OF THE DISCLOSURE

The present disclosure relates to a method for detecting at least one target nucleic acid sequence in a sample, comprising adding an RNA polymerase sequence to the target nucleic acid and detecting protons released by transcription activity, e.g. by an RNA polymerase.

### BACKGROUND

Quantitative real time polymerase chain reaction (qPCR) has become a standard for amplification of low amounts of DNA or RNA biomarkers. Most embodiments of qPCR and nucleic acid detection require fluorescently labelled sequence-specific probes or intercalating dyes. The exponential production of PCR products causes a concomitant exponential increase of fluorescence. In multiplex PCR settings, more than one target biomarker sequence within the same sample can be amplified in the qPCR reaction, by including multiple pairs of primers and different fluorescently labelled sequence-specific probes which can be detected simultaneously (e.g. using optical filters) or sequentially. However, the possibilities of highly multiplexed PCR methods is hampered by unavoidable spectral overlap between fluorescent dyes, and the limited segment of the spectrum for which affordable dyes exist for water-based solutions. Typically, in qPCR, the ability to multiplex targets using differently labelled probes is limited to the analysis of five target sequences in a single qPCR reaction.

The field of biochemical analysis is currently undergoing a miniaturization effort, with much attention given to the development of portable compact systems which integrate, often using microfluidics, operations that would previously have required a whole laboratory. Such microfluidic devices typically aim for processing tiny amounts of liquid and analytes, increasing multiplexing capabilities and allowing high throughput on a very low footprint, a fast turnaround time and low-cost approaches to the implemented biomolecular techniques.

Monitoring biological events such as nucleic acid hybridization with label-free biosensing methods based on field effect transistors (FETs) has attracted considerable attention, in particular because of their potential for miniaturization, easy integration into standard CMOS technologies and required electronics at a low cost, and multiplexing capabilities. Because methods based on FETs rely on an electrical rather than optical output signal (electrochemical detection), they are not affected by the limitations imposed by the choice of fluorophores and bulky equipment.

The ion sensitive field effect transistor (ISFET), sometimes simply referred to as a pH sensor, measures ion concentrations in a solution. Its suitability as readout for amplification reactions has been widely explored.

WO2003/073088 discloses a complementary metal-oxide semiconductor (CMOS)-based amplification device that has thermal actuation integrated with the reaction chemistry. An ISFET is used to monitor protons released in the pyrophosphate hydrolysis reaction associated with individual nucleotide insertion at the end of an oligonucleotide chain. Here, detection of individual nucleotide insertion by a pH sensitive ISFET is used in DNA sequencing technology based on “sequencing by synthesis”.

WO2008/107014 also relies on the fact that protons are a PCR product. qPCR may therefore also be achieved by using pH sensitive ISFETs for monitoring the protons released by each nucleotide insertion as amplification proceeds. The amplification is monitored by detecting a change of pH. The required degree of sensitivity in detection is achieved by carrying out the amplification in small volumes and low buffer capacity to ensure that the released protons lead to a rapid change in pH as the buffer capacity of the sample is overcome. The use of a DNA probe immobilized on the ISFET for capturing target DNA and separation of the target DNA from unwanted and interfering products is mentioned.

With little effort, scaling down ISFET designs to make arrays has resulted in more sensors packed on miniaturized chips. In terms of nucleic acid applications, large FET arrays for monitoring biological events have been described, in particular in the context of nucleic acid sequencing applications.

WO2010/138182 describes methods and apparatuses relating to FET arrays including large FET arrays for monitoring chemical and/or biological reactions such as nucleic acid sequencing-by-synthesis reactions. Some methods provided therein relate to improving signal (and also signal-to-noise ratio) from released hydrogen ions during nucleic acid sequencing reactions.

WO2010/047804 is directed to apparatus and chips comprising a large scale chemical field effect transistor arrays that include an array of sample-retaining regions capable of retaining a chemical or biological sample from a sample fluid for analysis. The apparatus and chips find their application in large scale pH-based DNA sequencing and other bioscience and biomedical applications.

In addition to its application in nucleic acid sequencing approaches, the utility of ISFETs for monitoring nucleic acid amplification has also been described in the context of CMOS chip platforms incorporating loop-mediated isothermal amplification (LAMP) and PCR.

Thus, Toumazou et al (2013, Nat Methods 10:641-646) used a standard CMOS process flow to create an integrated circuit that amplifies and simultaneously detects DNA on-chip using embedded heaters, 10 temperature sensors and 40 ISFET sensors. Conditions for LAMP and PCR were optimized under low buffering conditions while retaining amplification efficiency and specificity. The capability of multiplexing was demonstrated by interrogating two known biomarkers simultaneously.

Duarte-Guevara et al (2014, Anal Chem 86:8359-8367) investigated enhanced biosensing resolution in the context of a LAMP reaction with foundry-fabricated, individually addressable, dual-gated ISFETs.

Further, the applicability of the ISFET chip architecture described by Toumazou and Duarte-Guevara (supra) was tested in a clinical setting (Duarte-Guevara et al (2016), RSC Adv 6:103872-103887) wherein a dual-gate ISFET array platform was used for on-chip electrical detection of LAMP reactions that target food borne bacterial pathogens.

While the listed documents describe the ISFET approach as a workable biosensing technology that is automation friendly and provides a number of advantages, the disclosed methods still suffer drawbacks which limit their use in practice.

One such drawback is that electrochemical detection in a PCR requires a relatively long time for the amplification reaction to generate a sufficient amount of protons to get a measurable signal, especially at low concentrations of target nucleic acid. In this regard, Tomazou et al (supra) mention that a 40-cycle on-chip pH measuring PCR needs 35 min to be completed, which doesn't improve the turnaround time of traditional optically based PCR machines. In certain settings, e.g. point of care diagnostics, there is a need for not only a portable, compact system, but also to get test results as fast as possible without losing sensitivity, in order to allow for rapid intervention in patient management and outcome. Moreover, detection using ISFETs subjected to thermocycling (such as in PCR) may be challenging due to the temperature sensitivity of ISFETs, which needs to always be taken into account and compensated for.

Furthermore, current approaches to PCR with ISFETs as detection method often involve very specific assays targeting specific analytes, either by creating multiple microfluidic chambers with different primers injected in each of them, which increases the footprint in highly multiplexed settings, or with primers directly bound on the chip to initiate the reaction, which makes these chips unusable in scenarios where there is a need to include new targets in an assay. In this case, every time a new target needs to be added, a new chip must be produced to include probes for the new target, costing more effort from a production point of view. This is especially relevant for patient screening, where an all-in-one diagnostics approach is desirable (multiple analytes screened in one chip).

There remains a need for alternative nucleic acid analysis technologies.

### SUMMARY OF THE DISCLOSURE

One object of the disclosure is to overcome the limitations of prior biosensor-based detection methods.

Another object of the disclosure is to provide a method which generates a sufficient amount of protons for reliable detection of target nucleic acids.

Another object of the disclosure is to overcome the limitations of prior ISFET-based detection methods.

Another object of the disclosure is to enable the use of ISFET sensors without the need for high temperature conditions which affect test sensitivity and/or reliability.

Yet another object of the disclosure is to provide ways to detect target nucleic acid sequences in a way that is amenable to adaptation in a miniaturized and/or portable system.

Yet another object of the disclosure is to provide a method with a quick turn-around time to speed up existing technologies for the detection of target nucleic acid sequences, e.g. in a diagnostic or prognostic setting.

These objects, and others that are evident to the skilled person from the teachings herein, are met by the different aspects of the disclosure as defined herein and in the appended claims.

Thus, in a first aspect, the disclosure provides a method for detecting the presence of at least one target nucleic acid sequence in a sample, comprising the sequential steps of:
providing a sample suspected of containing the at least one target nucleic acid sequence;
adding an RNA polymerase promoter sequence to any target nucleic acid sequence present in the sample;
introducing the sample into a reaction chamber comprising
   at least one detection zone; and
   at least one capture nucleic acid arranged on a solid support and adapted to bind indirectly or directly to said target nucleic acid;
   to generate a nucleic acid sequence in single-stranded form, bound directly or indirectly to the capture nucleic acid arranged on the solid support;
applying elongation conditions which allow for generation of a nucleic acid strand complementary to said single-stranded nucleic acid to form a double-stranded nucleic acid comprising an RNA polymerase promoter sequence, bound directly or indirectly to the capture nucleic acid arranged on the solid support;
applying transcription conditions which allow for production of a transcript from the double-stranded nucleic acid captured on the solid support, whereby production of a transcript releases protons as transcription proceeds; and
detecting the presence of said protons as a signal from the detection zone, said signal being an indicator of the presence of the target nucleic acid sequence in the sample.

As used herein, the term “biological sample”, or simply “sample”, is intended to mean any one or more of a variety of biological sources that contain nucleic acid and/or cellular material, irrespective of whether it is freshly obtained from an organism (i.e. fresh tissue sample) or preserved by any method known in the art (e.g. an FFPE sample). Non-limiting examples of samples include cultures of cells, such as mammalian cells or eukaryotic microorganisms; body fluids; body fluid precipitates; lavage specimens; fine needle aspirates; biopsies; tissue samples; cancer cells; other types of cells obtained from a patient; cells from a tissue or in vitro cultured cells from an individual being tested and/or treated for disease or infection; or forensic samples. Non-limiting examples of body fluids include whole blood, bone marrow, cerebrospinal fluid (CSF), peritoneal fluid, pleural fluid, lymph fluid, serum, plasma, urine, chyle, stool, sperm, sputum, nipple aspirate, saliva, swab specimen, wash or lavage fluid and/or brush specimens.

The term “nucleic acid” and its equivalent “polynucleotide”, as used herein, refer to a polymer of ribonucleotides or deoxyribonucleotides bound together by phosphodiester linkages between the nucleotide monomers. The sequence which these bases (or their nucleosides, or the nucleotides of the latter) follow in a nucleic acid strand is termed “nucleic acid sequence” and is conventionally given in a so called 5′-end to 3′-end direction, referring to the chemical orientation of the nucleic acid strand. A sample suspected of containing the at least one target nucleic acid sequence is a sample suspected of containing target nucleic acid having the target nucleic acid sequence. Nucleic acids include, but are not limited to, DNA and RNA, including genomic DNA, mitochondrial or meDNA, cDNA, mRNA, rRNA, RNA, hnRNA, microRNA, lncRNA, siRNA, and various modified versions thereof. Nucleic acids will most commonly be obtained from natural sources, such as biological samples obtained from different types of organisms. On the other hand, nucleic acids can also be synthesized, recombined, or otherwise produced or engineered by known methods (e.g. PCR).

Regardless of whether the target nucleic acid binds directly or indirectly to the capture nucleic acid, the target nucleic acid is suitably introduced into the reaction chamber in single-stranded form. Methods to obtain single-stranded nucleic acid from double-stranded nucleic acid are well known by the skilled person, and may for instance include heating double-stranded nucleic acid at a temperature high enough (e.g. 90° C.) for it to become denatured, or may include chemical treatment of the double stranded target nucleic acid. Such treatment methods (denaturation conditions) can be applied before introduction of the sample into the reaction chamber, or alternatively, be applied within the reaction chamber prior to hybridizing any generated single-stranded target nucleic acid to the capture nucleic acid.

In one embodiment, the temperature of the reaction mixture comprising the target nucleic acid is within the range of 75-80° C. when the target nucleic acid is introduced. At this temperature, the single-stranded target nucleic acid hybridizes directly or indirectly to the capture nucleic acid. The product of hybridization of target and capture nucleic acids serves as a substrate for elongation conditions, so that the single-stranded part of the target nucleic acid is “filled in” to generate a double-stranded DNA of the entire target nucleic acid sequences and a functional, double-stranded RNA polymerase protomer sequence.

The “capture nucleic acid” arranged on a solid support suitable for use in the present invention may for instance be selected from the group consisting of DNA, RNA, PNA (peptide nucleic acid), LNA (locked nucleic acid), ANA (arabinonucleic acid) and HNA (hexitol nucleic acid). It may be an oligonucleotide which allows the formation of homoduplexes (DNA: DNA) or heteroduplexes with the target nucleic acid under appropriate hybridization conditions. As indicated in the described method, at least one nucleic acid sequence in single-stranded form is bound directly or indirectly to the capture nucleic acid arranged on the solid support. In this context, the term “bound” and “binding to” is equivalent to “hybridized to” and is meant to directly or indirectly capture the target nucleic acid for allowing surface-specific detection in proximity to a capture nucleic acid on the solid support. The capture portion of the capture nucleic acid (also called capture probe) may contain from 10 to 200 nucleotides, preferably from 15 to 50 nucleotides specific for the target or adapter nucleic acid sequence to be bound. Ideally, the capture nucleic acid contains 15, 16, 17, 19, 20, 21, 22, 23, 24, 25, 30, 35, 40, 45 or 50 nucleotides. The capture nucleic acid can also contain additional nucleotides that can act as a spacer between the capture portion and the solid surface or have a stabilizing function. The number of such additional nucleotides may be from 0 to 200 nucleotides, preferably from 0 to 50 nucleotides. Ideally, the spacer nucleic acid consists of 1, 5, 10, 15, 20, 25, 30, 35, 40, 45 or 50 nucleotides.

With “adding an RNA polymerase sequence to any target nucleic acid sequence present in the sample” is meant that RNA polymerase promoter sequence gets integrated in the target nucleic acid present in the sample. Various enzymatic methods suitable for this purpose are well known and include e.g. ligation techniques, recombination techniques and PCR (polymerase chain reaction).

With respect to the “RNA polymerase promoter sequence”, a person of ordinary skill in the art recognizes that this is a nucleotide sequence which is selectively recognized by an RNA polymerase and acts as a promoter for the attachment and activity of such an enzyme. Selecting and/or designing such a sequence suitable for use with a given specific RNA polymerase enzyme is within the capability of the skilled person. In one embodiment, the RNA polymerase promoter sequence has a length of from 17 to 26 base pairs. One example of a suitable RNA polymerase promoter sequence is a T7 RNA polymerase promoter sequence comprising 5′-TAATACGACTCACTATA-3′(SEQ ID NO:1). It may be preferred for an efficient promoter to also include at least one or more G nucleotide bases. Accordingly, another sequence used for T7 RNA transcription may comprise 5′-TAATACGACTCACTATAG-3′ (SEQ ID NO: 2).

After the double-stranded DNA has been generated under the elongation conditions, new conditions (transcription conditions) are applied to enable transcription from the RNA polymerase promoter sequence. In other words, under these conditions, RNA molecules are being synthesized in repeated initiation cycles, and one proton is released for every newly incorporated nucleotide. The isothermal and fast reaction will generate a local change in pH in a very short time, which can be measured in the detection zone, for example by a detection unit.

Thus, in the method of the first aspect, a target nucleic acid is provided with an RNA polymerase promoter sequence and immobilized via the capture nucleic acid on the solid support in the reaction chamber. After the target nucleic acid has been converted into double-stranded form, its presence may be detected through transcription using the transcription conditions. During transcription, protons are released and detected in the detection zone provided in the reaction chamber.

In this way, the method of the first aspect advantageously enables detection of a target nucleic acid separately from other steps, such as steps of amplification via PCR. One consequence of this is that the detection can be performed at isothermal conditions, e.g. at a constant, relatively low temperature such as room temperature, instead of at the high variable temperatures associated with thermocycling.

Another advantage of the method of the first aspect is that the transcription conditions may be adapted in such a way that a large amount of protons are released during a short time, ensuring efficient and reliable detection in the detection zone. Protons may be released directly, when NTPs are incorporated during transcription, or indirectly and simultaneously via conversion of released pyrophosphate.

Yet another advantage of the method is that it is suitable for processing of very small amounts of liquids and analytes. Another advantage is that the method is suitable for adaptation into a multiplex format, as described further below.

In one embodiment of the method, the detection zone comprises a detection unit used for detection of protons, i.e. for measuring a pH change. There are a variety of suitable technologies for measuring pH changes and thus detecting protons in the context of the present disclosure. pH changes may for example be measured using pH indicators such as fluorescence or solution absorbance. pH indicators which cause pH-dependent changes in the color of a solution and optical detection units for measuring such pH changes are well known to a skilled person. In a specific embodiment, the detection unit is an optical system in a reader. In another specific and advantageous embodiment, the detection unit is an ion sensitive field effect transistor (ISFET).

In one embodiment, the elongation conditions for converting single-stranded target nucleic acid into double-stranded nucleic acid comprise the presence of a DNA polymerase.

In one embodiment, the elongation conditions comprise a reaction temperature within the range of 75-90° C.

In one embodiment, the transcription conditions comprise the presence of an RNA polymerase. In a more specific embodiment, the RNA polymerase is the widely available T7 RNA polymerase.

In one embodiment, the transcription conditions comprise a reaction temperature within the range of 20-40° C. The reaction temperature of the transcription conditions may advantageously be around room temperature for optimal performance and durability of any detection unit, e.g. ISFET, arranged in the detection zone.

In one embodiment, the method of the first aspect of the disclosure is a method for detecting the presence of a plurality of target nucleic acid sequences, e.g. useful for monitoring several biomarkers that may be present in a patient sample. In such an embodiment, the plurality of target sequences is matched by a plurality of capture nucleic acids arranged on a solid support, and each of the capture nucleic acids is adapted to bind to a different target nucleic acid sequence.

In a more specific embodiment of this embodiment, the plurality of capture nucleic acids is arranged on the solid support in the form of an array, so that each capture nucleic acid represents an addressable location on the array. In such a set-up, the detection zone may, in an embodiment, be adapted to identify from which location or locations on the array a signal is, or signals are, detected.

In one embodiment of the method of the first aspect of the disclosure, the or each capture nucleic acid is designed so that its sequence matches the sequence of the target nucleic acid sequence that it is intended to capture. In this embodiment, at least a part of the sequence of the or each capture nucleic acid is identical or complementary to at least a part of the target nucleic acid sequence, such that it binds directly to one of the strands of the double-stranded nucleic acid to be detected.

FIG. 1 illustrates one embodiment of the method of the first aspect of the disclosure, in which two capture nucleic acids of different sequence are immobilized on two array addresses i and j of a solid support in a reaction chamber. Each capture nucleic acid is designed so that its sequence, or part of its sequence, matches (complements) part of the sequence of the target nucleic acid sequence that it is intended to capture. As shown at \textcircled{1} in FIG. 1, target nucleic acid in single-stranded form with added (integrated) RNA polymerase promoter sequence, hybridizes to the target-specific sequence of capture nucleic acid 1, and is thus immobilized at address i of the solid support. As shown at \textcircled{2}, elongation conditions are applied so that a double-stranded nucleic acid comprising an RNA polymerase sequence is formed using the single-stranded target nucleic acid as template for generation of a complementary strand. Finally, as shown at \textcircled{3}, transcription conditions, in this embodiment including an RNA polymerase, are applied, which leads to the production of several transcripts from the immobilized, double-stranded target sequence and the associated release of protons that are detected in a detection zone, e.g. by a detection unit such as an ISFET.

In an advantageous, alternative, embodiment, the at least one capture nucleic acid, for example that present at a given address on an addressable support, instead comprises a unique, non-target-derived, adapter sequence, and the method involves the use of adapter nucleic acid sequences to avoid the need to tailor the solid support for a given target nucleic acid to be detected. In this embodiment:
the step of adding an RNA polymerase promoter sequence further comprises adding a specific adapter sequence to any target nucleic acid sequence present in the sample;
the or each capture nucleic acid comprises a unique adapter sequence; and
the reaction chamber further comprises at least one adapter nucleic acid comprising
   a first, specific, adapter sequence which is identical or complementary to the specific adapter sequence(s) in the target nucleic acid sequence present in the sample; and
   a second, unique, adapter sequence which is complementary to the unique adapter sequence of said capture nucleic acid.

In this embodiment, the at least one capture nucleic acid, present at a given address in an addressable solid support, is unique in the sense that it does not directly match the sequence of the target nucleic acid directly. Neither is it identical to the corresponding unique adapter sequence at another address e.g. in an array.

In one embodiment, the specific adapter nucleic acid sequence added to any target nucleic acid sequence present in the sample may consist of at least a part of the nucleic acid sequence already present in the target nucleic acid. This will for example be the case when the specific adapter sequence is added through an amplification reaction such as e.g. PCR. In this case, the adapter nucleic acid sequence will be identical or complementary to the nucleic acid sequence of one of the primers used in the amplification reaction, and the same primer nucleic acid sequence will be identical or complementary to part of the target nucleic acid sequence.

In embodiments that use adapter nucleic acid in the reaction chamber, at least a part of the specific adapter sequence of the adapter nucleic acid is identical or complementary to at least a part of the corresponding nucleic acid sequence in any target nucleic acid present. At least another, unique, part of the nucleic acid sequence of the adapter nucleic acid is identical or complementary to at least a part of the capture nucleic acid sequence, such that it binds directly to one of the strands of the double-stranded nucleic acid to be detected.

With this design of capture and adapter sequences, the or each capture nucleic acid will bind indirectly to a corresponding target nucleic acid to be detected, via overlapping hybridization of the or each adapter nucleic acid to both the capture nucleic acid and the or each target nucleic acid. With this embodiment, it is possible to limit production to only one type of solid support (e.g. chip), which may be tailored for the detection of all possible target nucleic acid sequences through the design of suitable adapter sequences.

FIG. 2 illustrates one embodiment of the method of the first aspect of the disclosure, in which capture nucleic acids with different sequences are immobilized on two array addresses i and j of a solid support in a reaction chamber. Two different adapter nucleic acids are present in the reaction, each having a different unique adapter sequence for hybridizing to the corresponding capture nucleic acid sequence at a particular address, and each having a different target-specific sequence. Adapter nucleic acid 1 in FIG. 2 is specific for target nucleic acid 1, whereas adapter nucleic acid 2 has a specific sequence which is specific for another target nucleic acid not present in the reaction as illustrated. As shown at \textcircled{1} in FIG. 2, target nucleic acid in single-stranded form and with added RNA polymerase promoter sequence is added, hybridizes to the target-specific sequence of adapter nucleic acid 1, and is thus immobilized at address i of the solid support. As shown at \textcircled{2}, elongation conditions are applied so that a double-stranded nucleic acid comprising an RNA polymerase sequence is formed using the single-stranded target nucleic acid as template for generation of a complementary strand. Finally, as shown at \textcircled{3}, transcription conditions, in this embodiment including an RNA polymerase, are applied, which leads to the production of several transcripts from the immobilized, double-stranded target sequence and the associated release of protons that are detected in a detection zone, e.g. by a detection unit such as an ISFET.

As explained above, the method according to the first aspect of the disclosure involves adding an RNA polymerase promoter sequence to any target nucleic acid sequence present in the provided sample to be analyzed. In one embodiment of the disclosed method, this addition of an RNA polymerase promoter sequence is performed as part of an amplification reaction. Suitably, the amplification reaction is designed so as to amplify any target sequences present in a patient sample or other sample genetic material. As such, this embodiment bears resemblance to known amplification methods for detecting biomarkers or other sequence variants in a sample genetic material, but with the advantageous difference that the initial target amplification reaction is separate from the later step of target detection using the method of the first aspect of the disclosure. Additionally, the number of cycles of the amplification reaction does not have to be as many as the 35-40 cycles commonly used in amplification reactions, because the purpose of the reaction is only to add an RNA polymerase promoter sequence to any target nucleic acid present, without the requirement of measuring the amplification product as such.

In one embodiment using amplification to add RNA polymerase promoter sequence to a target nucleic acid, the amplification reaction comprises the sequential steps of:
providing a sample genetic material;
denaturing the sample genetic material;
adding at least one pair of target primers under conditions allowing annealing of the primers to the sample genetic material; said pair of target primers comprising
   a first primer comprising
      a sequence specific for the target sequence; and
      an RNA polymerase promoter sequence; and
   a second primer comprising
      a sequence specific for the target sequence;
         said sequences specific for the target sequence in said primers being selected so as to enable amplification of a target nucleic acid sequence when said target nucleic acid sequence is present in the sample genetic material;
carrying out an amplification reaction for a predetermined number of cycles, resulting in the amplification of, and addition of an RNA polymerase promoter sequence to, any target nucleic acid sequence present in the sample genetic material.

As used herein, the term “primer” refers to a nucleic acid of a defined oligonucleotide length, which, when forming a duplex with a polynucleotide template, is capable of acting as a point of initiation of nucleic acid synthesis and being extended from its 3′ end along the template so that an extended double strand (duplex) is formed. As used herein, the term “primer pair” refers to at least two such primers, one being termed the “forward primer” and the other one the “reverse primer” complementary to the nucleotide sequences flanking, i.e. being at the beginning and the end, respectively, of that section of the template nucleic acid that one desires to amplify.

As will be readily understood by a skilled person, the amplification reaction in this embodiment utilizes at least one primer pair flanking a target region of interest, such as illustrated schematically in FIG. 3.

The amplification reaction in this embodiment may be adapted to the various different embodiments, described above, of carrying out the ensuing detection of the presence of target (interchangeably referred to as the “detection stage”).

Thus, in an embodiment in which the detection stage is designed to detect the presence of a plurality of different target nucleic acid sequences, a preceding amplification reaction is suitably designed to include a plurality of different primer pairs, each pair being adapted to amplify each target nucleic acid sequence to be detected. In the amplification reaction in this embodiment, the addition of at least one pair of target primers comprises addition of a plurality of pairs of target primers, each individual primer pair comprising sequences that are specific for different target sequences, so that said polymerase chain reaction results in the amplification of all different target sequences that are present in the sample genetic material.

In one embodiment, the amplification reaction is used to also add those sequences to the target nucleic acid which enable the target nucleic acid to be bound directly or indirectly to the capture nucleic acid arranged on the solid support.

In an embodiment of the detection stage in which the or each capture nucleic acid is identical or complementary to at least a part of the target nucleic acid sequence, such that it binds directly to one of the strands of the double-stranded nucleic acid to be detected, the or each second primer in the amplification reaction comprises a sequence which is identical or complementary to at least a part of the sequence of the or each capture nucleic acid. Amplification using such a second primer will incorporate a sequence into the amplified target nucleic acid which will hybridize to the capture nucleic acid, when the amplified target nucleic acid is introduced in single-stranded form into the reaction chamber for detection.

In one embodiment, the second primer may comprise sequence which is identical or complementary to target sequences, and will thus add such target-specific nucleic acid sequences to the amplified target nucleic acid. In this embodiment of the detection stage, each capture nucleic acid is designed to have a nucleic acid sequence which is identical or complementary to at least a part of the target-specific nucleic acid sequence, allowing the amplified target nucleic acid to hybridize to the capture nucleic acid.

Alternatively, the second primer may comprise non-target specific nucleic acid sequences in addition to the sequence which is identical or complementary to target sequences, and will thus add also such non target-specific nucleic acid sequences to the amplified target nucleic acid. In this embodiment of the detection stage, each capture nucleic acid is designed to have a nucleic acid sequence which is identical or complementary to at least a part of the non-target specific nucleic acid sequence, allowing the amplified target nucleic acid to hybridize to the capture nucleic acid.

In an alternative embodiment, using the more flexible capture-adapter-target concept discussed in connection with the detection stage above, the amplification reaction is instead used to introduce a sequence complementary to the adapter sequence into the target nucleic acid by designing the or each second primer accordingly. In this embodiment, then, the or each second primer further comprises a specific adapter sequence, whereas the or each capture nucleic acid comprises a unique adapter sequence. The reaction chamber further comprises at least one adapter nucleic acid comprising a first, specific, adapter sequence which is identical to the specific adapter sequence(s) in the or each second primer; and a second, unique, adapter sequence which is complementary to the unique adapter sequence of said capture nucleic acid, such that the or each capture nucleic acid binds indirectly to a corresponding target nucleic acid, via overlapping hybridization of the or each adapter nucleic acid to both the capture nucleic acid and the or each second primer. Thus, advantageously, in an embodiment where a plurality of target sequences are investigated, it is possible in this case to add different adapter sequences to different target nucleic acids, so that each specific target nucleic acid to be detected comprises its own adapter sequence, useful for binding to only the adapter nucleic acid which comprises that specific adapter sequence. All adapter nucleic acids also comprise a unique adapter sequence which is complementary only to the unique sequence on the corresponding capture nucleic acid.

In a more specific embodiment of this advantageous set-up, a further advantage may be obtained by designing the adapter nucleic acid to include additional target sequence adjacent to the first, specific, adapter sequence (i.e. adjacent to that stretch of sequence which is also present in the or each second primer). In this way, the stretch of complementarity between the target nucleic acid and the adapter nucleic acid is increased when the target nucleic acid is indirectly bound to the capture nucleic acid, because there is additional matching sequence between the amplified target sequence and the adapter sequence, beyond the portion of target sequence provided by the or each second primer. The additional advantage of such a design is that the annealing between target and adapter nucleic acids can then be carried out at a higher temperature than that which is necessary for primer hybridization. As a result, interference from excess primers in the hybridization between target and adapter nucleic acids is suppressed or eliminated. In addition, the existence of a target part in the adapter nucleic acid which is slightly longer than the target sequence in the or each second primer also adds selectivity against unwanted byproducts (for example primer dimers) when applying elongation conditions to synthesize the complementary strand of the single-stranded target nucleic acid in the detection stage (FIG. 1, step \textcircled{2}).

In one embodiment, the amplification reaction is a polymerase chain reaction (PCR).

Carrying out an amplification reaction (e.g. PCR) on a sample genetic material in order to prepare the target nucleic acid for later detection has the benefits of allowing the addition, to the target nucleic acid, of RNA polymerase promoter sequences and sequences designed for direct or indirect binding to the capture nucleic acid, through introduction of these sequence elements into the or each primer pair used for amplification of one or more specific desired target sequences in the sample genetic material. Another benefit of carrying out amplification (e.g. PCR) prior to detection in this way, is that reagents in the amplification reaction mix, such as DNA polymerase and dNTPs, may be useful components in the elongation step of the detection stage. In this embodiment, the reaction mix from the amplification reaction may simply be transferred to the reaction chamber of the detection stage without additional manipulation and used directly. However, the method of this embodiment also benefits from the fact that amplification and detection are separated into distinct reaction steps, taking place sequentially and in different reaction conditions. As such, the amplification reaction does not have to be sustained for so long that it in itself generates a detectable number of protons, but only long enough to amplify sufficient amounts of target nucleic acid with RNA polymerase promoter sequences added. This means that analysis may be carried out quickly and efficiently. Likewise, the detection of protons, e.g. using temperature-sensitive ISFETs, is not subjected to the high and varying temperatures associated with thermocycling in amplification methods such as PCR, with the consequence that equipment and detection units will have a longer life-span and higher reliability. In this way, major draw-backs of previously known detection methods based on pH sensors are avoided.

In one embodiment in which amplification is used to add the required sequences (i.e. RNA polymerase promoter sequence and any additional sequences for direct or indirect binding to capture nucleic acid), the predetermined number of cycles in the amplification reaction may be only one cycle, which will nevertheless fulfil the function of adding the sequences to any target nucleic acids present in the sample genetic material. In one embodiment, the predetermined number of cycles is from 1 to 40 cycles, such as from 1 to 30 cycles, such as from 1 to 20 cycles, such as from 1 to 10 cycles, such as from 2 to 10 cycles. In specific embodiments, the predetermined number of cycles is 2, 3, 4, 5, 6, 7, 8, 9 or 10 cycles.

The methods described herein can be automated. Thus, in a preferred embodiment of the disclosure, any method of the disclosure is performed by an automated system. As used herein, the term “automated system” may refer to an integrated platform comprising an instrument and disposable materials, such as plastics and solutions, which the system uses in an automated manner to complete a certain process. While such a process can be initiated by a user, no user intervention is required during the automated processing within the system until after completion of the process. As used herein, the term “instrument” is to be understood as a machine equipped with at least a user interface (e.g. comprising at least a start button or an electricity plug), a computer with software, programmed to perform functions such as running an assay using the method of the present disclosure. This may involve e.g. mixing, heating, data detection, data collection, data analysis etc. In a preferred embodiment, the disposable material is provided in the form of a kit. As used herein, the term “kit” is to be interpreted as a set comprising at least one article or as an assembly of articles or equipment needed for a specific purpose, for example to perform a molecular biology method or assay. A second aspect of the present disclosure provides a kit comprising a reaction chamber having a detection zone for detecting proton release/accumulation during transcription of nucleic acid, capture nucleic acid arranged on a solid support and adapted to bind indirectly or directly target nucleic acid, and reagents for applying conditions for elongation and transcription of nucleic acid.

Also provided here are uses of the methods and products (such as kits and automated systems) described herein for detecting at least one target nucleic acid sequence in a sample, comprising adding an RNA polymerase sequence to the target nucleic acid and detecting protons released by transcription activity, e.g. RNA polymerase activity.

A third aspect of the disclosure provides various in vitro methods, in which the presence, absence or amount of target nucleic acid in a sample is used as a basis for a clinically relevant decision concerning a subject.

In one embodiment, the method is an in vitro diagnostic method, comprising a further step of using the presence, absence or amount of at least one target nucleic acid in the sample as a basis for determining a diagnosis of a condition in a subject.

In another embodiment, the method is an in vitro prognostic method, comprising a further step of using the presence, absence or amount of at least one target nucleic acid in the sample as a basis for determining a prognosis of a condition in a subject.

In yet another embodiment, the method is an in vitro subject stratification method, comprising the further step of using the presence, absence or amount of at least one target nucleic acid in the sample as a basis for predicting the likelihood of success of a treatment of a condition in a subject.

In a related embodiment, the method is an in vitro subject stratification method, comprising the further step of using the presence, absence or amount of at least one target nucleic acid in the sample as a basis for predicting the likelihood of resistance to a treatment of a condition in a subject.

In another embodiment, the method is an in vitro method for selecting a suitable treatment of a condition in a subject, comprising the further step of using the presence, absence or amount of at least one target nucleic acid in the sample as a basis for selecting a suitable treatment of the condition in the subject.

Preferred embodiments of such methods may comprise the steps of
obtaining a sample genetic material from a subject to be tested, or supplying a sample genetic material previously obtained;
enriching any target nucleic acid present in said sample genetic material to render it detectable by a method as disclosed herein.

In a particular embodiment of such methods, detection is performed using a method in which an amplification reaction is used to add an RNA transcription promoter sequence, and enrichment is performed using said amplification reaction with primers designed to amplify said target nucleic acid from said sample genetic material.

In certain embodiments of such methods, said sample genetic material is obtained from a sample taken from the subject. As defined above, a sample may be selected from the group consisting of cultures of cells, body fluids, body fluid precipitates, lavage specimens, fine needle aspirates, biopsies, tissue samples, cancer cells, other types of cells obtained from a subject, cells from a tissue or in vitro cultured cells from a subject being tested and/or treated for disease or infection, and forensic samples. In the case of a body fluid sample, the body fluid may be selected from the group consisting of whole blood, bone marrow, cerebrospinal fluid (CSF), peritoneal fluid, pleural fluid, lymph fluid, serum, plasma, urine, chyle, stool, sperm, sputum, nipple aspirate, saliva, swab specimen, wash or lavage fluid and brush specimens.

In certain embodiments of such methods, the subject is a mammal. In particular embodiments, the subject is a human being.

Also provided are kits for diagnosis and/or prognosis and/or subject stratification for treatment and/or the selection of a suitable treatment of a subject. Such kits may incorporate any of the various features, aspects and embodiments mentioned in connection with the various disclosed methods and uses.

It is considered to be within the skill of a person of skill in the arts of modern biochemistry and gene technology to design and implement the various nucleic acid elements used in the various aspects of the present disclosure, once such skilled person is provided with the general principles disclosed herein. As an example, a skilled person will be able to select suitable target sequences to detect in a sample genetic material and the associated primer pairs for detection and amplification of such target sequences, including the length of the target-specific sequence in the primers and other design considerations. Likewise, a skilled person can design suitable capture nucleic acid sequences for immobilization on the solid support, and, when applicable, suitable adapter nucleic acid sequences having the requisite degree of overlap with target and capture nucleic acid sequences, respectively. Furthermore, in embodiments employing amplification to add the necessary sequence elements to target nucleic acids, it is within the capability of the skilled person to select the proper reaction parameters of such amplification, such as reaction temperatures and components of the reaction mix, including the nature and concentration of polymerase enzymes, dNTPs or NTPs, and other known factors.

Also, while the invention has been described with reference to various exemplary aspects and embodiments, it will be understood by those skilled in the art that various changes may be made and equivalents may be substituted for elements thereof without departing from the scope of the disclosure herein. In addition, many modifications may be made to adapt a particular situation to the teachings of the invention without departing from the essential scope thereof. Therefore, it is intended that the invention not be limited to any particular embodiment contemplated, but that it include all embodiments falling within the scope of the appended claims.

### BRIEF DESCRIPTION OF THE FIGURES

FIG. 1 is a schematic illustration of one embodiment of a method of the disclosure, in which capture nucleic acids are immobilized on two array addresses i and j of a solid support in a reaction chamber and any target nucleic acid present in a sample is directly bound to capture nucleic acid.

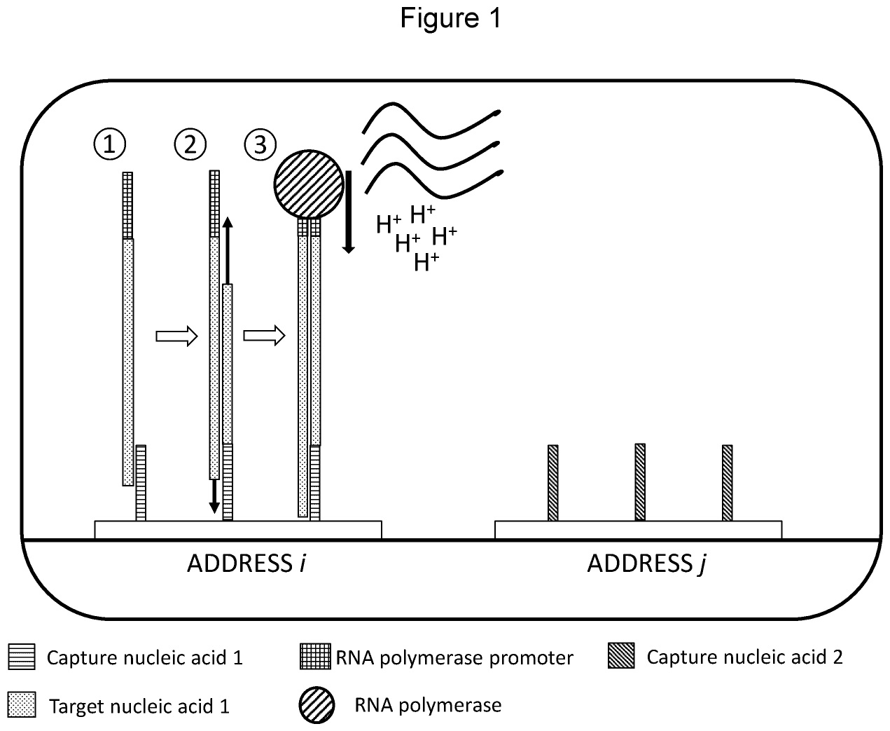

FIG. 2 is a schematic illustration of another embodiment of a method of the disclosure, in which capture nucleic acids are immobilized on two array addresses i and j of a solid support in a reaction chamber and any target nucleic acid present in a sample is indirectly bound to capture nucleic acid via adapter nucleic acid.

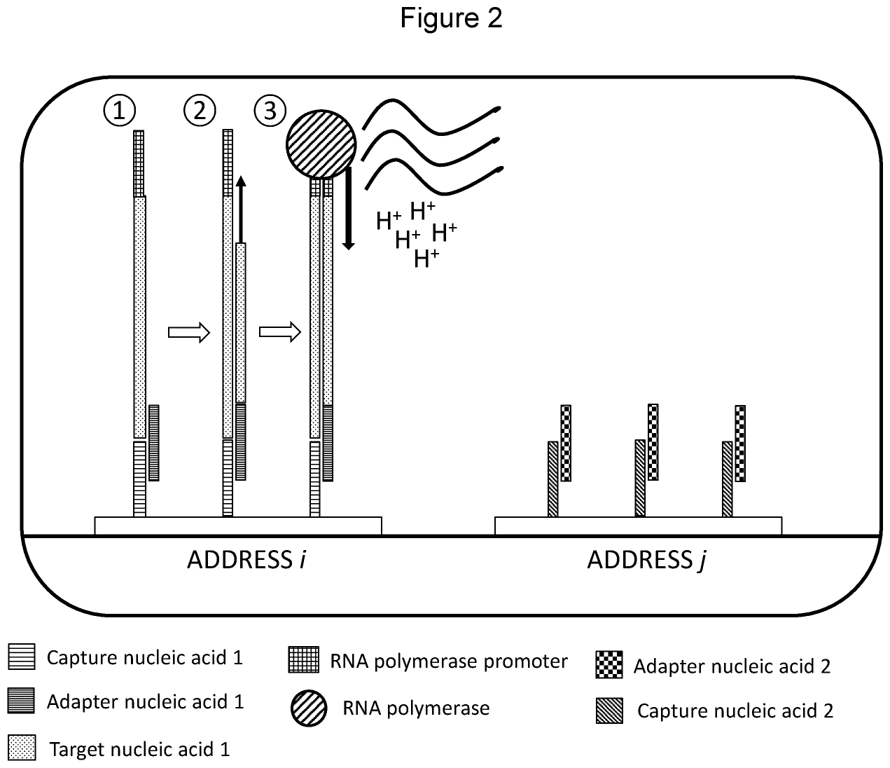

FIG. 3 is a schematic illustration of a primer design useful in one embodiment of a method of the disclosure, in which amplification is used to add an RNA polymerase promoter sequence to a target nucleic acid to be detected.

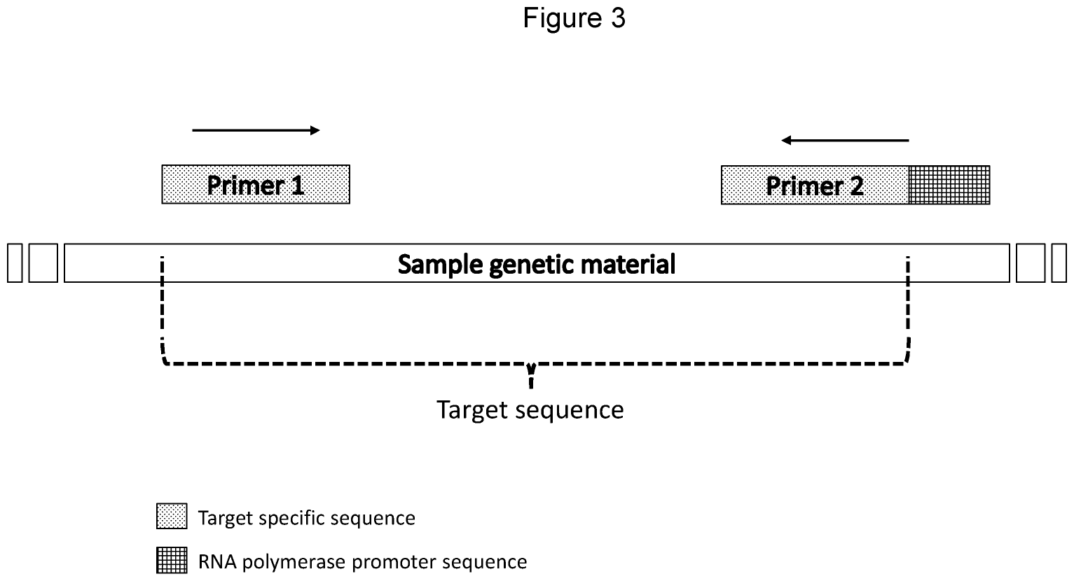

FIG. 4 is a diagram showing the current (y axis) measured from reaction of different concentrations (x axis) of template target nucleic acid in the experiment described in Example 1.

FIG. 5 is a photograph of an ISFET sensor array having capture nucleic acid immobilized using click chemistry as described in Example 2.

FIG. 6 shows diagrams of output current (y axis) vs time (x axis) in the presence (A) or absence (B) of target sequence, after baseline subtraction from the four sensors depicted in the inset of FIG. 6A, and as described in Example 2.

### EXAMPLES

The following Examples illustrate embodiments of the disclosure, put into practice in different settings.

### Example 1

An ISFET sensor array (Taiwan Semiconductor Manufacturing Company; TSMC) was used to detect the pH change coming from T7 RNA polymerase activity over a model target nucleic acid including a T7 promoter sequence. The array had a dimension of 850×850 μm² and contained 1024 sensors with hafnium oxide (HfO₂) as the pH sensitive layer. The sensors were arranged in 32 rows and 32 columns. The arrays were produced by standard Complementary Metal Oxide Semiconductor (CMOS) processing and bonded on a printed circuit board providing electrical connection to a measuring station (referred as Demobox), which was in turn connected to a laptop running control software also provided by TSMC. A polymeric confinement well was used to confine the buffer over the sensing area to a volume of 20 μl. A leakless miniature Ag/AgCl reference electrode (eDAQ) connected to the Demobox was used to bias the buffer.

The target nucleic acid used in the experiment was the pSP73 DNA plasmid, known to comprise a T7 RNA polymerase promoter sequence upstream of a multiple cloning site. A mixture of the plasmid, a CutSmart® 10× buffer and the restriction enzyme HPAI in RNAse free water was heated at 37° C. for 1 h to allow the plasmid to be cut at a specific sequence and linearized. All reagents were provided by Integrated DNA Technologies (IDT). Subsequently, a T7 RNA polymerase master mix was prepared in CHES buffer, to a final composition of 20 mM NaCl, 6 mM MgCl₂, 10 Mm DTT, 1 Mm spermidine, 12 U/μl of T7 enzyme and 0.5 mM of each NTP. The pH was adjusted to a value of around 8 with NaOH. The ISFET sensor array chip was tested for pH sensitivity in 1 M CHES buffer at different pH values between 7.5 and 8.5 in steps of 0.2 before the experiment.

The target nucleic acid prepared as described above was added to the T7 RNA polymerase master mix at different concentrations for a final volume of 20 μl and quickly poured with the help of a pipette into the confinement well glued onto the ISFET sensor array chip connected to the Demobox.

The software was programmed to measure the output current from all the sensors in the array every second. Multiple experiments were conducted in the same way for different concentrations of the target nucleic acid. The T7 RNA polymerase buffer only was also measured, as a negative control.

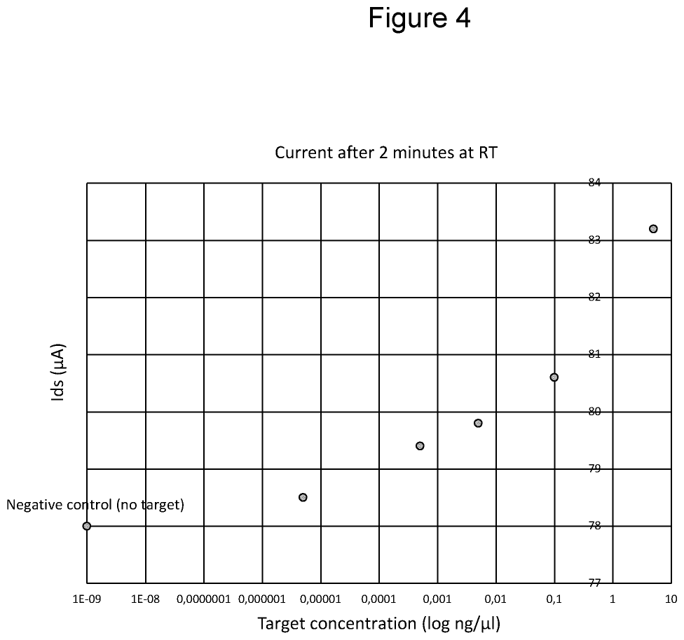

Results are shown in FIG. 4. The diagram shows the value of output current (absolute values) measured after 2 minutes from the beginning of the experiment (i.e. from the moment the buffer was placed over the chip) vs the logarithm of the target concentration on a representative sensor. Because different concentrations of target will provide different pH changes, also the measured current should vary depending on pH. Specifically, because the ISFETs are n-channel transistors, the current is expected to decrease with decreasing target concentration (less protons generated). The lowest concentration of linearized plasmid tested was of 10⁻⁶ ng/μl (approximately 10⁴ molecules) which was still recognized by the sensor when compared to the negative control.

### Example 2

A proof of concept system was tested and designed, with three oligonucleotides acting as capture probe, adapter and target nucleic acid, respectively. Oligonucleotides were dissolved in IDTE buffer, pH 8, at concentrations of 100 μM.

TABLE 1
Nucleic acid sequences used for spotting test

| | | | |
|---|---|---|---|
| | | | SEQ |
| | 5′ | | ID |
| Designation | modification | Sequence | NO: |

Capture probes

| | | | |
|---|---|---|---|
| 5capture_01 | /5DBCOTEG/ | TTTGACGGCTGCGCAGACCA | 3 |
| 5capture_02 | /5DBCOTEG/ | TTTTGCCCGACGCGTCTGGT | 4 |
| 5capture_03 | /5DBCOTEG/ | TTTCGGGTCCGGAGGTGCGT | 5 |
| 5capture_04 | /5DBCOTEG/ | TTTCACCAGGTCCGCAGCCA | 6 |

Test probes

| | | | |
|---|---|---|---|
| 3capture_01_HEX | /5HEX/ | TGGTCTGCGCAGCCGTC | 7 |
| 3capture_02_FAM | /56-FAM/ | ACCAGACGCGTCGGGCA | 8 |
| 3capture_03_FAM | /56-FAM/ | ACGCACCTCCGGACCCG | 9 |
| 3capture_04_FAM | /56-FAM/ | TGGCTGCGGACCTGGTG | 10 |

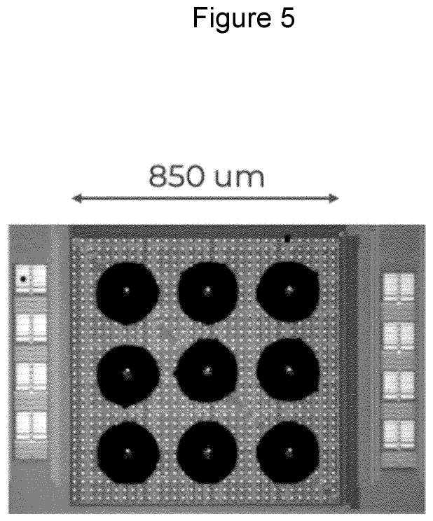

To test the set-up, four different capture probes (Table 1; SEQ ID NO: 3-6) were immobilized on the ISFET sensor array using click chemistry (Movilli et al (2020), ACS Langmuir 36:4272-4279). In a first step, chips obtained from TSMC were wet and dry cleaned with solvents and mild ozone. Then, they were functionalized with 3-azidopropyltriethoxysilane (Gelest) for 6 h at 70° C., providing azide groups on the surface which can react efficiently with DBCO modified oligonucleotides by click chemistry. The DBCO functionalized capture probes were designed and spotted with a SciFLEXARRAYER SX (Scienion) on the azide functionalized chips at a concentration of 10 μM in 1 nl of a buffer made of 1 M NaCl and 10 mM Tris HCl at pH 8. The spotted chips were incubated in a high humidity environment (85%) to avoid evaporation, and the click chemistry reaction was allowed to last for 1 h. After 1 h, the chips were rinsed with deionized water. FIG. 5 is a photograph of an array spotted with capture probes.

A test to check for successful click chemistry reaction was performed as follows. Four different test probes (Table 1; SEQ ID NO:7-10), complementary to the spotted capture probes and carrying a fluorescent molecule, were allowed to hybridize on the spotted chip at room temperature (RT) for 1 h. Then, fluorescence was observed through an optical microscope equipped with excitation/emission filters. It was established that the optical signal came from the spotted areas, meaning the hybridization of test probes on the respective spotted capture probes had been successful.

TABLE 2
Nucleic acid sequences used for proof of concept

| SEQ | | | | |
|---|---|---|---|---|
| ID | | 5′ | | |
| NO: | Designation | modification | Tm | Sequence |
| 3 | Capture | /5DBCOTEG/ | 73 | TTTGACGGCTGCGCAGACCA |
| 11 | Adapter | /5DBCOTEG/ | 73 | GTAATTAATACGACTCACTAT |
| | | | | AGGGAGATTTTCAGTGTGAG |
| | | | | ATGGTCTGCGCAGCCGTC |
| 12 | Target | | | TCTCACACTGAAAATCTCCCT |
| | | | | ATAGTGAGTCGTATTAATTAC |

After the successful test had been carried out, adapter (SEQ ID NO:11) and target (SEQ ID NO: 12) nucleic acid molecules were hybridized to the capture probe (SEQ ID NO:3) spotted on the chip (Table 2). The sequences of the adapter and target oligonucleotides were designed such that, once hybridized, they would be double-stranded and have a T7 RNA polymerase promoter sequence on the side exposed to the buffer. The adapter and target oligonucleotides were allowed to hybridize only on a specific spot over the array by complementarity with the capture probe at that location, while there was no hybridization on spots without capture probes or with non-complementary capture probes. A solution of the two oligonucleotides at 500 nM each in 1 M NaCl, 10 mM Tris HCl buffer was prepared, pipetted directly onto the ISFET sensor array and left to sit for 1 h at RT. After 1 h, the chip was washed with the same solution. At this point, the ISFET sensor chip was ready to be tested using the set-up described in Example 1. A confinement well for the buffer was attached and the chip inserted into the Demobox, programmed to measure the output current from all sensors in the array over time. A T7 RNA polymerase master mix having the same composition as that described in Example 1 was used.

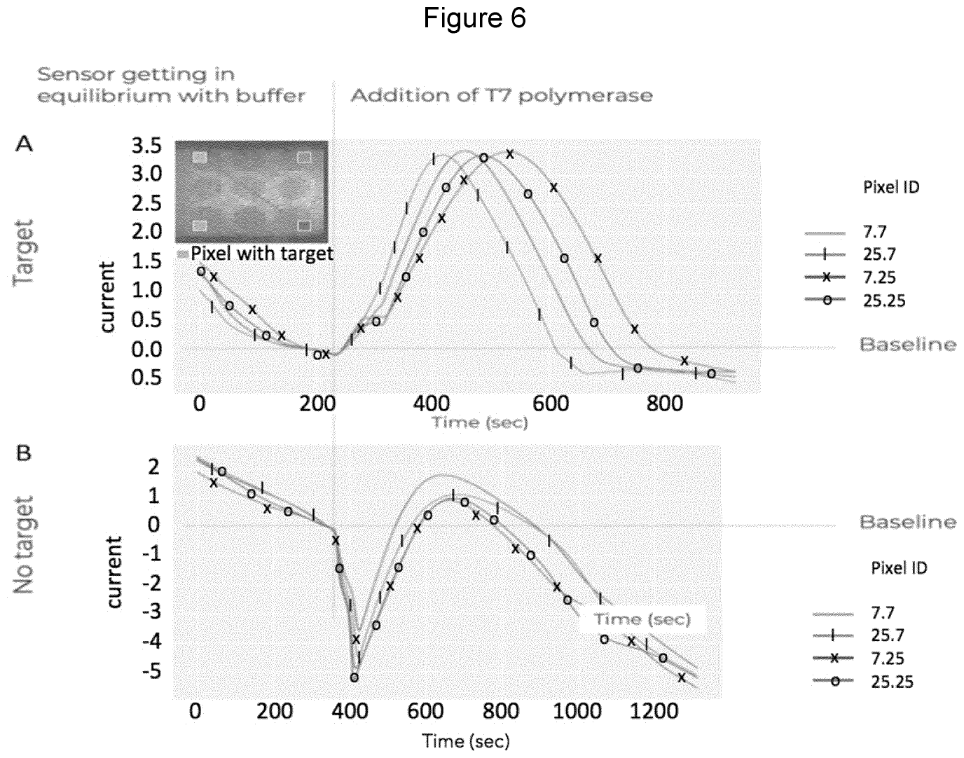

FIG. 6 shows the output current from each sensor, mirroring the spot pattern. The chemical composition on the spotted and not spotted locations is different, with the area outside the spots having only a silanization layer with the azide group and the spots having also oligonucleotides on top of this layer, and hence also the electrochemical potential causing a current value.

FIGS. 6A and 6B show graphs of current vs time after baseline subtraction of the four sensors highlighted in the inset. The sensor/curve pair is coded as indicated in the legend. At first, a T7 RNA polymerase master mix without the enzyme was placed over the chip for stabilizing the electrical output signal. Under these conditions, the reaction could not start because the enzyme is missing. Subsequently, this enzyme-free buffer was pipetted out of the well and replaced with a buffer containing all the components necessary for the reaction to start.

As shown in FIG. 6A, the pixel where the target nucleic acid (comprising a T7 promoter sequence) was hybridized (upper left corner, pixel 7.7) shows a faster transient response upon introduction of the T7 RNA polymerase master mix containing the enzyme, compared to the pixels lacking the target. The reason for the faster response is thought to be that the reaction starts over sensors having hybridized target comprising T7 RNA polymerase promoter sequence, so the protons generated upon nucleotide insertion are immediately detected by the ISFET. Eventually however, diffusion over the entire chip generates a response also from sensors located at a distance.

As a negative control, a time response was also obtained from a chip which was similarly spotted with capture probes, but had no target nucleic acid hybridized on it. The current vs time response of this chip after baseline subtraction is shown in FIG. 6B. Here, no delay between the response of pixels at different locations was seen, which is attributed to the fact that no reaction takes place and all sensors are simultaneously exposed to the same chemical buffer composition (electrochemical potential).

### Itemized Listing of Embodiments

1. Method for detecting the presence of at least one target nucleic acid sequence in a sample, comprising the sequential steps of:
providing a sample suspected of containing the at least one target nucleic acid sequence;
adding an RNA polymerase promoter sequence to any target nucleic acid sequence present in the sample;
introducing the sample into a reaction chamber comprising
   at least one detection zone; and
   at least one capture nucleic acid arranged on a solid support and adapted to bind indirectly or directly to said target nucleic acid;
   to generate a nucleic acid sequence in single-stranded form, bound directly or indirectly to the capture nucleic acid arranged on the solid support;
applying elongation conditions which allow for generation of a nucleic acid strand complementary to said single-stranded nucleic acid to form a double-stranded nucleic acid comprising an RNA polymerase promoter sequence, bound directly or indirectly to the capture nucleic acid arranged on the solid support;
applying transcription conditions which allow for production of a transcript from the double-stranded nucleic acid captured on the solid support, whereby production of a transcript releases protons as transcription proceeds; and
detecting the presence of said protons as a signal from the detection zone, said signal being an indicator of the presence of the target nucleic acid sequence in the sample.

2. Method according to item 1 for detecting the presence of a plurality of target nucleic acid sequences, wherein said plurality of target sequences is matched by a plurality of capture nucleic acids arranged on a solid support, each adapted to bind indirectly or directly to a different target nucleic acid sequence.

3. Method according to item 2, wherein said plurality of capture nucleic acids is arranged in the form of an array on said solid support, so that each capture nucleic acid represents an addressable location on the array.

4. Method according to item 3, wherein a detected signal from the detection zone is identified as originating from a specific addressable location on the array.

5. Method according to any preceding item, wherein at least a part of the sequence of the or each capture nucleic acid is identical or complementary to at least a part of the target nucleic acid sequence, such that it binds directly to one of the strands of the double-stranded nucleic acid to be detected.

6. Method according to any one of items 1-4, wherein
the step of adding an RNA polymerase promoter sequence further comprises adding a specific adapter sequence to any target nucleic acid sequence present in the sample;
the or each capture nucleic acid comprises a unique adapter sequence; and
said reaction chamber further comprises at least one adapter nucleic acid comprising
   a first, specific, adapter sequence which is identical or complementary to the specific adapter sequence(s) in the target nucleic acid sequence present in the sample; and
   a second, unique, adapter sequence which is complementary to the unique adapter sequence of said capture nucleic acid;
such that the or each capture nucleic acid binds indirectly to a corresponding target nucleic acid to be detected, via overlapping hybridization of the or each adapter nucleic acid to both the capture nucleic acid and the or each target nucleic acid.

7. Method according to any preceding item, wherein the steps of providing a sample and adding an RNA polymerase promoter sequence and, when present, a specific adapter sequence, are performed as part of an amplification reaction.

8. Method according to item 7, wherein said amplification reaction comprises the sequential steps of:
providing a sample genetic material;
denaturing the sample genetic material;
adding at least one pair of target primers under conditions allowing annealing of the primers to the sample genetic material; said pair of target primers comprising
   a first primer comprising
      a sequence specific for the target sequence; and
      an RNA polymerase promoter sequence; and
   a second primer comprising
      a sequence specific for the target sequence;
         said sequences specific for the target sequence in said primers being selected so as to enable amplification of a target nucleic acid sequence when said target nucleic acid sequence is present in the sample genetic material;
carrying out an amplification reaction for a predetermined number of cycles, resulting in the amplification of, and addition of an RNA polymerase promoter sequence to, any target nucleic acid sequence present in the sample genetic material.

9. Method according to item 8, wherein the addition of at least one pair of target primers comprises addition of a plurality of pairs of target primers, each individual primer pair comprising sequences that are specific for different target sequences, so that said amplification reaction results in the amplification of all different target sequences that are present in the sample genetic material.

10. Method according to any one of items 7-9, wherein at least a part of the sequence of the or each capture nucleic acid is identical or complementary to the or each second primer, such that it binds directly to one of the strands of the or each target nucleic acid.

11. Method according to any one of items 7-9, wherein
the or each second primer further comprises a specific adapter sequence;
the or each capture nucleic acid comprises a unique adapter sequence; and
said reaction chamber further comprises at least one adapter nucleic acid comprising
   a first, specific, adapter sequence which is identical to the specific adapter sequence(s) in the or each second primer; and
   a second, unique, adapter sequence which is complementary to the unique adapter sequence of said capture nucleic acid;
      such that the or each capture nucleic acid binds indirectly to a corresponding target nucleic acid, via overlapping hybridization of the or each adapter nucleic acid to both the capture nucleic acid and the or each second primer.

12. Method according to item 11, wherein said at least one adapter nucleic acid further comprises additional target sequence adjacent to the first, specific, adapter sequence, such that the stretch of complementarity between the desired amplicon and the adapter nucleic acid is increased beyond the portion of target sequence provided by the second primer.

13. Method according to any one of items 7-12, wherein said amplification reaction is a polymerase chain reaction (PCR).

14. Method according to any preceding item, wherein said detection zone comprises a detection unit, for example an ion sensitive field effect transistor.

15. Method according to any preceding item, wherein said elongation conditions comprise a reaction temperature within the range of 75-90° C.

16. Method according to any preceding item, wherein said elongation conditions comprise the presence of a DNA polymerase.

17. Method according to any preceding item, wherein said transcription conditions comprise a reaction temperature within the range of 20-40° C.

18. Method according to any preceding item, wherein said transcription conditions comprise the presence of an RNA polymerase.

19. Method according to any preceding item, wherein said RNA polymerase promoter sequence is a T7 RNA polymerase promoter sequence.

20. Method according to any one of items 18-19, wherein said RNA polymerase is a T7 RNA polymerase.

21. Method according to any preceding item, which is an in vitro diagnostic, prognostic, patient condition stratification or treatment selection method, comprising a further step of using the presence, absence or amount of at least one target nucleic acid in the sample as a basis for determining a diagnosis of a condition in a subject, or for determining a prognosis of a condition in a subject, or for determining a stratification of a patient, or for determining a selection of treatment of a patient, respectively.

22. Method according to item 21, comprising:
obtaining a sample genetic material from a subject to be tested;
enriching any target nucleic acid present in said sample genetic material to render it detectable by a method according to any one of items 1-18.

23. Method according to item 22, in which said detection is performed using a method according to any one of items 7-13, and enrichment is performed using said amplification reaction with primers designed to amplify said target nucleic acid from said sample genetic material.

24. Method according to any one of items 21-23, wherein said sample genetic material is obtained from a sample taken from the subject.

25. Method according to item 24, wherein said sample is selected from the group consisting of cultures of cells, body fluids, body fluid precipitates, lavage specimens, fine needle aspirates, biopsies, tissue samples, cancer cells, other types of cells obtained from a subject, cells from a tissue or in vitro cultured cells from a subject being tested and/or treated for disease or infection, and forensic samples.

26. Method according to item 25, wherein said sample is a body fluid selected from the group consisting of whole blood, bone marrow, cerebrospinal fluid (CSF), peritoneal fluid, pleural fluid, lymph fluid, serum, plasma, urine, chyle, stool, sperm, sputum, nipple aspirate, saliva, swab specimen, wash or lavage fluid and brush specimens.

27. Method according to any one of items 21-26, wherein the subject is a mammal, for example a human being.

1. A method for detecting the presence of at least one target nucleic acid sequence in a sample, comprising the sequential steps of:
providing a sample suspected of containing the at least one target nucleic acid sequence;
adding an RNA polymerase promoter sequence to any target nucleic acid sequence present in the sample;
introducing the sample into a reaction chamber comprising
at least one detection zone; and
at least one capture nucleic acid arranged on a solid support and adapted to bind indirectly or directly to said target nucleic acid;
to generate a nucleic acid sequence in single-stranded form, bound directly or indirectly to the capture nucleic acid arranged on the solid support;
applying elongation conditions which allow for generation of a nucleic acid strand complementary to said single-stranded nucleic acid to form a double-stranded nucleic acid comprising an RNA polymerase promoter sequence, bound directly or indirectly to the capture nucleic acid arranged on the solid support;
applying transcription conditions which allow for production of a transcript from the double-stranded nucleic acid captured on the solid support, whereby production of a transcript releases protons as transcription proceeds; and
detecting the presence of said protons as a signal from the detection zone, said signal being an indicator of the presence of the target nucleic acid sequence in the sample.

2. The method according to claim 1 for detecting the presence of a plurality of target nucleic acid sequences, wherein said plurality of target sequences is matched by a plurality of capture nucleic acids arranged on a solid support, each adapted to bind indirectly or directly to a different target nucleic acid sequence.

3. The method according to claim 2, wherein said plurality of capture nucleic acids is arranged in the form of an array on said solid support, so that each capture nucleic acid represents an addressable location on the array.

4. The method according to claim 1, wherein at least a part of the sequence of the or each capture nucleic acid is identical or complementary to at least a part of the target nucleic acid sequence, such that it binds directly to one of the strands of the double-stranded nucleic acid to be detected.

5. The method according to claim 1, wherein
the step of adding an RNA polymerase promoter sequence further comprises adding a specific adapter sequence to any target nucleic acid sequence present in the sample;
the or each capture nucleic acid comprises a unique adapter sequence; and
said reaction chamber further comprises at least one adapter nucleic acid comprising
a first, specific, adapter sequence which is identical or complementary to the specific adapter sequence(s) in the target nucleic acid sequence present in the sample; and
a second, unique, adapter sequence which is complementary to the unique adapter sequence of said capture nucleic acid;
such that the or each capture nucleic acid binds indirectly to a corresponding target nucleic acid to be detected, via overlapping hybridization of the or each adapter nucleic acid to both the capture nucleic acid and the or each target nucleic acid.

6. The method according to claim 1, wherein the steps of providing a sample and adding an RNA polymerase promoter sequence and, when present, a specific adapter sequence, are performed as part of an amplification reaction.

7. The method according to claim 6, wherein said amplification reaction comprises the sequential steps of:
providing a sample genetic material;
denaturing the sample genetic material;
adding at least one pair of target primers under conditions allowing annealing of the primers to the sample genetic material; said pair of target primers comprising
a first primer comprising
a sequence specific for the target sequence; and
an RNA polymerase promoter sequence; and
second primer comprising
a sequence specific for the target sequence;
said sequences specific for the target sequence in said primers being selected so as to enable amplification of a target nucleic acid sequence when said target nucleic acid sequence is present in the sample genetic material;
carrying out an amplification reaction for a predetermined number of cycles, resulting in the amplification of, and addition of an RNA polymerase promoter sequence to, any target nucleic acid sequence present in the sample genetic material.

8. The method according to claim 7, wherein the addition of at least one pair of target primers comprises addition of a plurality of pairs of target primers, each individual primer pair comprising sequences that are specific for different target sequences, so that said amplification reaction results in the amplification of all different target sequences that are present in the sample genetic material.

9. The method according to claim 6, wherein at least a part of the sequence of the or each capture nucleic acid is identical or complementary to the or each second primer, such that it binds directly to one of the strands of the or each target nucleic acid.

10. The method according to claim 6, wherein
the or each second primer further comprises a specific adapter sequence;
the or each capture nucleic acid comprises a unique adapter sequence; and
said reaction chamber further comprises at least one adapter nucleic acid comprising
a first, specific, adapter sequence which is identical to the specific adapter sequence(s) in the or each second primer; and
a second, unique, adapter sequence which is complementary to the unique adapter sequence of said capture nucleic acid;
such that the or each capture nucleic acid binds indirectly to a corresponding target nucleic acid, via overlapping hybridization of the or each adapter nucleic acid to both the capture nucleic acid and the or each second primer.

11. The method according to claim 10, wherein said at least one adapter nucleic acid further comprises additional target sequence adjacent to the first, specific, adapter sequence, such that the stretch of complementarity between the desired amplicon and the adapter nucleic acid is increased beyond the portion of target sequence provided by the second primer.

12. The method according to claim 6, wherein said amplification reaction is a polymerase chain reaction (PCR).

13. The method according to claim 1, wherein said detection zone comprises a detection unit, for example an ion sensitive field effect transistor.

14. The method according to claim 1, wherein said elongation conditions comprise the presence of a DNA polymerase.

15. The method according to claim 1, wherein said transcription conditions comprise the presence of an RNA polymerase.

16. The method according to claim 1, wherein said RNA polymerase promoter sequence is a T7 RNA polymerase promoter sequence.

17. The method according to claim 1, which is an in vitro diagnostic, prognostic, patient condition stratification or treatment selection method, comprising a further step of using the presence, absence or amount of at least one target nucleic acid in the sample as a basis for determining a diagnosis of a condition in a subject, or for determining a prognosis of a condition in a subject, or for determining a stratification of a patient, or for determining a selection of treatment of a patient, respectively.

18. (canceled)

19. (canceled)


SEQUENCE LISTING (12 sequences)

SEQ ID NO: 1 - DNA, 17 bp, synthetic construct
  misc_feature 1..17: note = Engineered promoter sequence
  source 1..17: mol_type = other DNA
taatacgactcactata

SEQ ID NO: 2 - DNA, 18 bp, synthetic construct
  misc_feature 1..18: note = Engineered promoter sequence
  source 1..18: mol_type = other DNA
taatacgactcactatag

SEQ ID NO: 3 - DNA, 20 bp, synthetic construct
  misc_feature 1..20: note = Engineered capture probe
  source 1..20: mol_type = other DNA
tttgacggctgcgcagacca

SEQ ID NO: 4 - DNA, 20 bp, synthetic construct
  misc_feature 1..20: note = Engineered capture probe
  source 1..20: mol_type = other DNA
ttttgcccgacgcgtctggt

SEQ ID NO: 5 - DNA, 20 bp, synthetic construct
  misc_feature 1..20: note = Engineered capture probe
  source 1..20: mol_type = other DNA
tttcgggtccggaggtgcgt

SEQ ID NO: 6 - DNA, 20 bp, synthetic construct
  misc_feature 1..20: note = Engineered capture probe
  source 1..20: mol_type = other DNA
tttcaccaggtccgcagcca

SEQ ID NO: 7 - DNA, 17 bp, synthetic construct
  misc_feature 1..17: note = Engineered test probe
  source 1..17: mol_type = other DNA
tggtctgcgcagccgtc

SEQ ID NO: 8 - DNA, 17 bp, synthetic construct
  misc_feature 1..17: note = Engineered test probe
  source 1..17: mol_type = other DNA
accagacgcgtcgggca

SEQ ID NO: 9 - DNA, 17 bp, synthetic construct
  misc_feature 1..17: note = Engineered test probe
  source 1..17: mol_type = other DNA
acgcacctccggacccg

SEQ ID NO: 10 - DNA, 17 bp, synthetic construct
  misc_feature 1..17: note = Engineered test probe
  source 1..17: mol_type = other DNA
tggctgcggacctggtg

SEQ ID NO: 11 - DNA, 59 bp, synthetic construct
  misc_feature 1..59: note = Engineered adapter sequence
  source 1..59: mol_type = other DNA
gtaattaatacgactcactatagggagattttcagtgtgagatggtctgcgcagccgtc

SEQ ID NO: 12 - DNA, 42 bp, synthetic construct
  misc_feature 1..42: note = Engineered test target
  source 1..42: mol_type = other DNA
tctcacactgaaaatctccctatagtgagtcgtattaattac